(19) United States
(12) Patent Application Publication
(10) Pub. No.: US 2023/0398365 A1
(43) Pub. Date: Dec. 14, 2023

(54) SYSTEM AND METHOD FOR ELECTRODE IMPLANTATION

(71) Applicant: Cochlear Limited, Macquarie University, NSW (AU)

(72) Inventors: Peter Gibson, Macquarie University, NSW (AU); Nicholas Charles Kendall Pawsey, Macquarie University (AU); Paul Michael Carter, Macquarie University (AU)

(21) Appl. No.: 18/250,344

(22) PCT Filed: Oct. 15, 2021
(86) PCT No.: PCT/IB2021/059538

§ 371 Date: Apr. 24, 2023

Related U.S. Application Data
(60) Provisional application No. 63/114,421, filed on Nov. 16, 2020.

(51) Int. Cl. A61N 1/375; A61N 1/05; A61N 1/36

(52) U.S. Cl. CPC A61N 1/37518; A61N 1/0541; A61N 1/36039

(57) ABSTRACT

A method includes providing an electrode configured to be implanted within a body portion of a recipient in tissue at a target implantation location, the electrode comprising a screw portion. The method further includes driving the screw portion into the tissue at the target implantation location, monitoring at least one signal indicative of insertion of the screw portion into the tissue, and stopping the driving in response to the at least one signal being indicative of a predetermined insertion of the screw portion.

### BACKGROUND

### Field

The present application relates generally to systems and methods for facilitating implantation of an electrode of a medical device on or within a recipient's body, and more specifically to electrodes configured to stimulate the auditory and/or vestibular systems.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, a method comprises providing an electrode configured to be implanted within a body portion of a recipient in tissue at a target implantation location, the electrode comprising a screw portion. The method further comprises driving the screw portion into the tissue at the target implantation location. The method further comprises monitoring at least one signal indicative of insertion of the screw portion into the tissue during said driving. The method further comprises stopping said driving in response to the at least one signal being indicative of a predetermined insertion of the screw portion.

In another aspect disclosed herein, an apparatus comprises a bone screw portion configured to be rotated about an axial direction to drill and/or tap into bone tissue outside and adjacent to a vestibular cavity of a recipient. The apparatus further comprises a head portion configured to be mechanically engaged and rotated about the axial direction to drill and/or tap the bone screw portion into the bone tissue. The apparatus further comprises an electrically conductive connector portion configured to be in electrical communication with an electrically conductive conduit while the bone screw portion and the head portion are rotated about the axial direction.

In another aspect disclosed herein, a system comprises an electrode comprising a screw portion configured to be inserted into bone tissue of a recipient. The system further comprises a controller in electrical communication with the electrode. The controller is configured to monitor a status of the screw portion during insertion of the screw portion into the bone tissue and to transmit electrical stimulation signals to the electrode after the screw portion is inserted into the bone tissue.

### BRIEF DESCRIPTION OF THE DRAWINGS

Implementations are described herein in conjunction with the accompanying drawings, in which:

FIG. 1A shows a cross-sectional view cut in the mid-modiolar axial plane of the vestibular system with an example position suitable for placement of a bone screw of a vestibular electrode for stimulating the otoliths in accordance with certain implementations described herein;

FIG. 1B schematically illustrates an axial computed tomography view of the vestibular system with a dashed oval indicating an example position suitable for placement of a bone screw of a vestibular electrode for stimulating the otoliths in accordance with certain implementations described herein;

FIG. 1C schematically illustrates a view of the vestibular system from a direction along an example drilling line that is posterior to the facial nerve and with the dura anterior to the example drilling line in accordance with certain implementations described herein;

FIGS. 2A and 2B are flow diagrams of examples method in accordance with certain implementations described herein

FIGS. 3A-3C schematically illustrates an example driving system and apparatus that are compatible with the example methods of FIGS. 2A-2B and is configured to provide vestibular stimulation in accordance with certain implementations described herein;

FIGS. 4A-4B schematically illustrate example apparatus comprising a bone screw portion and a head portion in accordance with certain implementations described herein;

FIG. 5A schematically illustrates two views of an example apparatus comprising a fixture in accordance with certain implementations described herein;

FIG. 5B schematically illustrates the apparatus of FIG. 5A implanted into tissue in accordance with certain implementations described herein;

FIG. 5C schematically illustrates two views of an example electrically conductive connector of the apparatus in accordance with certain implementations described herein;

FIG. 5D schematically illustrates two views of a connector in mechanical and electrical communication with the fixture in accordance with certain implementations described herein;

FIGS. 6A-6D schematically illustrate another example apparatus in accordance with certain implementations described herein;

FIGS. 7A-7D schematically illustrate another example apparatus comprising a second component in accordance with certain implementations described herein; and

FIG. 8 is a flow diagram of an example method 800 for implanting the apparatus 400 in accordance with certain implementations described herein.

### DETAILED DESCRIPTION

The vestibular system is a portion of the inner ear which enables the sensation of angular and linear motion. Neural signals corresponding to this sensed motion are used by the brain to assist in a variety of processes including balance and determining orientation, and in related motor activities such as walking, standing, and visual orientation.

Various dysfunctions and abnormalities of the vestibular system are known, and in severe cases they can result in significant disability for those so afflicted. In older persons, the loss of stability attendant upon vestibular dysfunction can lead to a greatly increased likelihood of a fall, and consequent loss of independence and mobility. Meniere's disease is an abnormality of the vestibular system which affects approximately 1 in 2000 people worldwide. Meniere's disease has symptoms that are highly variable between patients, and it can be relatively difficult to diagnose with certainty. The symptoms of Meniere's disease include but are not limited to: periodic episodes of rotary vertigo or dizziness; fluctuating, progressive, unilateral or bilateral hearing loss; unilateral or bilateral tinnitus; and a sensation of fullness or pressure in one or both ears.

Approximately 85% of affected people affected by Meniere's disease can be treated with measures such as medication, dietary changes, lifestyle changes, or behavioral therapy. The remaining 15% of affected people are not assisted sufficiently by these measures, and typically turn to one of a variety of surgical procedures. A vestibular stimulator system can be configured to provide electrical stimulations (e.g., using electrodes external to the recipient's body or implanted on or within the recipient's body) in order to treat vestibular disease.

For example, a vestibular prosthesis is under development to restore balance by electrical stimulation of an electrode placed inside the vestibule via a hole drilled in the stapes footplate, with the aim of providing a solution for people who have both bilateral vestibular dysfunction and deafness, so there is no disadvantage to opening the vestibule. However, there is a population of people with bilateral vestibular dysfunction who still have functional hearing to various levels, and it can be desirable to provide these people with a solution that reduces (e.g., minimizes) the risk of hearing loss by placing the stimulation electrode sufficiently near to the vestibular nerve without breaching the cochleovestibular system. See e.g., A. Ramos et al., “Chronic Electrical Stimulation of the Otolith Organ: Preliminary Results in Humans with Bilateral Vestibulopathy and Sensorineural Hearing Loss,” Spec. Ed. New Concepts in Electrical Stimulation in Vestibular Dysfunction, Audiology & Neurotology, doi:10.1159/000503600 (2020).

Accessing the vestibular nerve through a retrosigmoid approach is a challenging surgery which involves extra risk by virtue of opening into the brain space. Approaching the area directly through the semicircular canals is also challenging since it is very difficult to manually drill through the bony labyrinth without breaching the semicircular canals. It can also be difficult to stop drilling at precisely the correct point without drilling into the vestibule.

Certain implementations described herein provide a minimally invasive technique to access an inner ear and/or middle ear region of a recipient's body to implant a stimulation electrode sufficiently close to the cochleovestibular system to provide stimulation signals to a target portion of the cochleovestibular system (e.g., the vestibule; vestibular nerve; cochlea) without breaching the cochleovestibular system and adversely affecting a hearing capability of the recipient. The implantation technique can monitor at least one signal indicative of insertion of the electrode (e.g., indicative of force and/or torque applied to a bone screw portion of the electrode; indicative of electrical impedance between the electrode and the body) during implantation.

Certain implementations described herein are configured to provide at least one stimulation electrode configured to treat vestibular dysfunction (e.g., in recipients with hearing; in recipients without hearing). Other certain implementations described herein are configured to provide at least one stimulation electrode configured to treat tinnitus in recipients with hearing (e.g., placed on the promontory of the cochlea; see, e.g., U.S. Pat. Appl. Publ. No. 2019/0167985). Still other certain implementations described herein are configured to provide at least one ground electrode and/or stimulation electrode configured to be used in an auditory prosthesis (e.g., as part of a cochlear implant). For example, the at least one ground electrode and/or stimulation electrode can be placed in the apex of the cochlea, in the base of the cochlea, or near the cochlea nerve to create specific current paths and electric fields for stimulation of the auditory nerve (see, e.g., U.S. Pat. Appl. Publ. No. 2018/0369571).

In certain implementations, the at least one stimulation electrode is configured to be part of a stimulation system configured to operate automatically (e.g., automatically enabling and/or disabling stimulation based on signals indicative of symptoms or the imminent onset of symptoms; operating in continuous “stimulating” mode to maintain a manageable level of function in cases of severe dysfunction). In certain other implementations, the at least one stimulation electrode is part of a stimulation system configured to operate in response to input signals from the recipient (e.g., when the recipient determines that they are experiencing symptoms to be treated and/or in a preventative mode in which the recipient seeks to prevent the onset of an attack), and/or in response to input signals from a medical practitioner. An example stimulation system compatible with certain implementations described herein is disclosed by K. Hageman et al., “Design of a Vestibular Prosthesis for Sensation of Gravitoinertial Acceleration,” J. Med. Devices, Vol. 10, pp. 030923-1 (2016).

The teachings detailed herein are applicable, in at least some implementations, to any type of implantable medical device (e.g., implantable sensory prostheses) configured to apply stimulation signals to a portion of the recipient's body (e.g., cochlea; vestibule). The implantable medical device of certain implementations described herein comprises a first portion (e.g., external to a recipient) and a second portion (e.g., implanted on or within the recipient), the first portion configured to wirelessly transmit power and/or data to the second portion. For example, the implantable medical device can comprise an auditory prosthesis system utilizing an external sound processor configured to transcutaneously provide power to an implanted assembly (e.g., comprising an actuator). In certain such examples, the external sound processor is further configured to transcutaneously provide data (e.g., control signals) to the implanted assembly that responds to the data by generating stimulation signals that are perceived by the recipient as sounds. Examples of auditory prosthesis systems compatible with certain implementations described herein include but are not limited to: electro-acoustic electrical/acoustic systems, cochlear implant devices, implantable hearing aid devices, middle ear implant devices, Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of medical device that can utilize the teachings detailed herein and/or variations thereof.

Merely for ease of description, apparatus and methods disclosed herein are primarily described with reference to an illustrative medical device, namely a vestibular implant. However, the teachings detailed herein and/or variations thereof may also be used with a variety of other medical devices that provide a wide range of therapeutic benefits to recipients, patients, or other users. In some implementations, the teachings detailed herein and/or variations thereof can be utilized in other types of implantable medical devices beyond vestibular devices (e.g., vestibular implants). For example, apparatus and methods disclosed herein and/or variations thereof may also be used with one or more of the following: auditory prostheses; visual devices (e.g., bionic eyes); visual prostheses (e.g., retinal implants); sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; pain relief devices; etc. The concepts described herein and/or variations thereof can be applied to any of a variety of implantable medical devices comprising an implanted component configured to use magnetic induction to receive power (e.g., transcutaneously) from an external component and to store at least a portion of the power in at least one power storage device (e.g., battery). The implanted component can also be configured to receive control signals from the external component (e.g., transcutaneously) and/or to transmit sensor signals to the external component (e.g., transcutaneously) while receiving power from the external component.

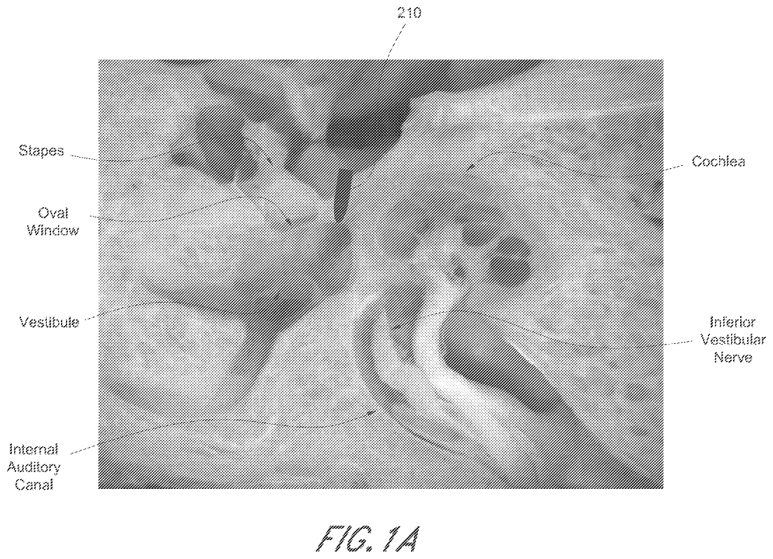

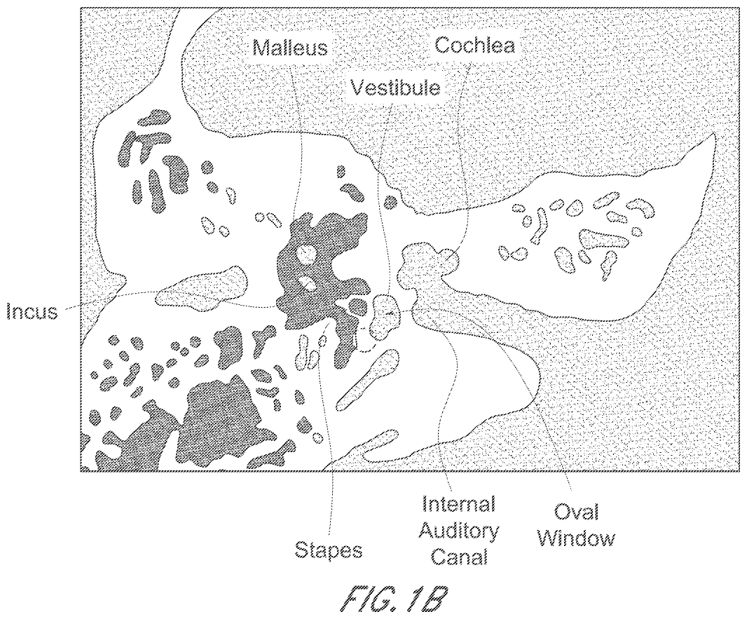

FIGS. 1A-IC show various views of the vestibular system in an inner ear region of a recipient. FIG. 1A shows a cross-sectional view cut in the mid-modiolar axial plane of the vestibular system with an example position suitable for placement of a bone screw of a vestibular electrode for stimulating the otoliths in accordance with certain implementations described herein. FIG. 1B schematically illustrates an axial computed tomography view of the vestibular system with a dashed oval indicating an example position suitable for placement of a bone screw of a vestibular electrode for stimulating the otoliths in accordance with certain implementations described herein. FIG. 1C schematically illustrates a view of the vestibular system from a direction along an example drilling line that is posterior to the facial nerve and with the dura anterior to the example drilling line in accordance with certain implementations described herein. The vestibular system includes the vestibule (e.g., vestibular cavity) that houses the three semicircular canals (anterior, posterior, and horizontal) and the otolithic organs (saccule and utricle). The three semicircular canals are arranged substantially orthogonal to each other and are filled with endolymph fluid. Upon rotation of the head with a component of motion in the appropriate direction, movement of the fluid within the canal is detected by hair bundles connected to hair cells, and stimulation of the hair cells cause by the fluid movement produces a corresponding neural signal in nerve fibers.

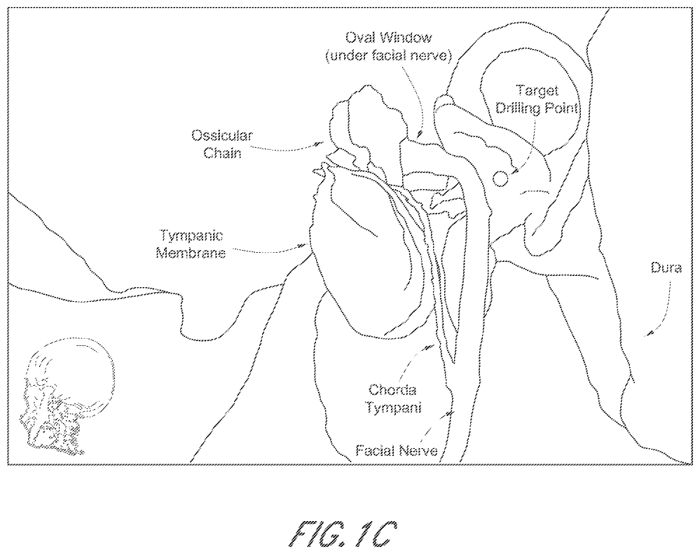

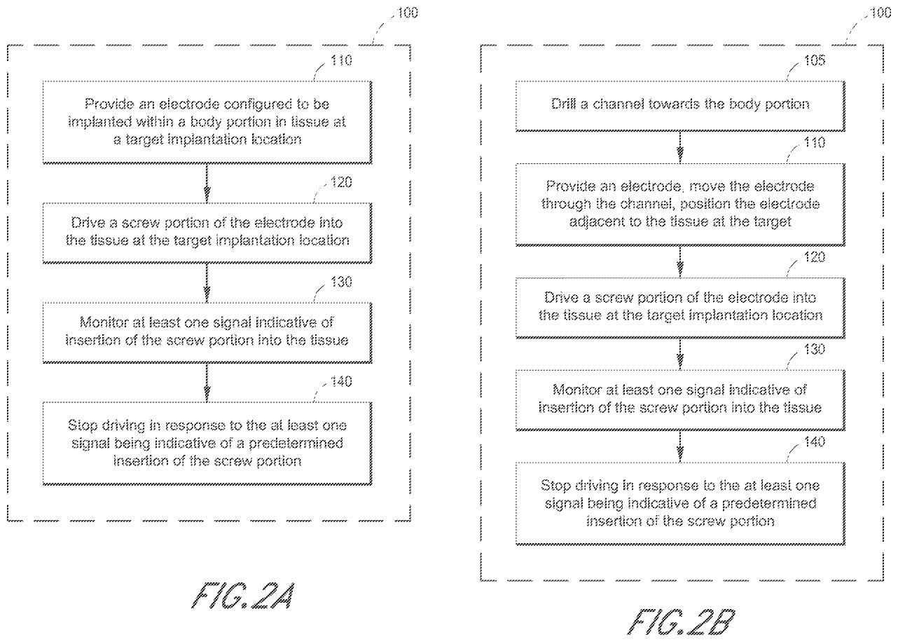

FIGS. 2A and 2B are flow diagrams of examples method 100 in accordance with certain implementations described herein. FIGS. 3A-3C schematically illustrates an example driving system 300 and apparatus 400 that are compatible with the example methods 100 of FIGS. 2A-2B and is configured to provide vestibular stimulation in accordance with certain implementations described herein. In certain implementations, the apparatus 400 comprises an electrode 210 comprising a screw portion 212 configured to be screwed into the bone portion and a connection portion 214 configured to extend outwardly from the bone portion and to be in electrical communication with a stimulation system configured to provide stimulation signals to the electrode 210. The screw portion 212 and the connection portion 214 can comprise an electrically conductive and biologically compatible material (e.g., titanium; titanium alloy).

In an operational block 110, the method 100 comprises providing an electrode 210 configured to be implanted within a body portion of a recipient in tissue at a target implantation location. In certain implementations, the body portion comprises a middle ear region and/or an inner ear region of the recipient. As schematically illustrated in FIGS. 3A-3C, the tissue at the target implantation location can comprise bone tissue outside and adjacent to a vestibular cavity (e.g., vestibule) of the recipient, and the target implantation location can be sufficiently close to the otoliths and/or the vestibular nerve such that the otoliths and/or vestibular nerve are stimulated by a predetermined voltage and/or current applied to the electrode 210. In certain other implementations in which the electrode 210 is configured to provide cochlear stimulation (e.g., as part of a cochlear implant and/or a tinnitus treatment device), the target implantation location is on a surface portion of a cochlea within the middle ear region, such that nerves within the cochlear are stimulated by a predetermined electrical current and/or voltage applied to the electrode 210.

In certain implementations, as shown in FIG. 2B, the method 100 comprises drilling a channel towards the body portion along a pre-operatively determined path in an operational block 105, and providing the electrode 210 in the operational block 110 further comprises moving the electrode 210 through the channel and positioning the electrode 210 adjacent to the tissue at the target implantation location. The path can be configured to avoid damaging predetermined tissue portions. In certain implementations, a specific drilling path can be planned pre-operatively (e.g., based on pre-operative imaging), and drilling the channel can be guided by a navigation guidance system (e.g., commercially available) to avoid damaging the selected structures. In certain implementations, a hand-guided robotic drill that is configured to drill through bone towards a cavity and to stop drilling without breaching the cavity can be used. For example, in certain implementations in which the target implantation location is within a middle ear region and/or an inner ear region of the recipient, said drilling the channel comprises drilling toward the vestibule or vestibular nerve and stopping prior to breaching into the vestibular cavity (e.g., avoiding risk of hearing loss) whilst avoiding damaging the otolithic organs within the vestibular cavity, the vestibular nerve, other selected structures in the inner ear or middle ear regions (e.g., avoiding damaging any semicircular canal, facial nerve, and/or cochleovestibular nerve of the recipient). Example drilling systems compatible with certain implementations described herein are disclosed by X. Du et al., “Robustness analysis of a smart surgical drill for cochleostomy,” Int. J. Med. Robot., Vol. 9 (1) pp. 119-126.21 (2013) and P. Brett et al., “Feasibility Study of a Hand Guided Robotic Drill for Cochleostomy,” Biomed. Res. Int'l, Vol. 2014, Article ID 656325, 7 pages, https://dx.doi.org/10.1155/2014/656325. While such drilling systems have been disclosed for creating a cochleostomy for insertion of a cochlear electrode, they can be used for creating an opening for a vestibular electrode in accordance with certain implementations described herein.

For example, the example drilling line schematically illustrated by FIG. 1C is along a direction that is posterior to the facial nerve and anterior to the dura in accordance with certain implementations described herein. The insert at the bottom left portion of FIG. 1C shows the skull orientation of the view of FIG. 1C. The white dot in FIG. 1C is over the vestibule, just posterior to the oval window which is partly obscured by the facial nerve in FIG. 1C. As schematically illustrated by FIG. 1C, by imaging the cochleovestibular system and using path planning and intraoperative navigation, a drilling trajectory can be followed in accordance with certain implementations described herein to reach the vestibule and implant the electrode 210 without compromising other structures (e.g., the dura, the facial nerve).

In an operational block 120, the method 100 further comprises driving (e.g., inserting) the screw portion 212 into the tissue at the target implantation location (e.g., pressing the screw portion 212 against the tissue and rotating the screw portion 212 about an axial direction of the screw portion 212). In certain implementations, the screw portion 212 is configured to be self-drilling (e.g., the screw portion 212 is configured to create a hole in the tissue upon the screw portion 212 being driven into the tissue) and/or self-tapping (e.g., the screw portion 212 is configured to form hole threads in the tissue upon the screw portion 212 being driven into the tissue). Thus, in certain implementations in which the screw portion 212 is both self-drilling and self-tapping, after being driven into the tissue, the screw portion 212 resides in the hole and is mated with the hole threads, with the connection portion 214 extending outwardly from the hole. In certain other implementations, the tissue at the target implantation location comprises a previously-drilled pilot hole configured to at least partially receive the screw portion 212. In certain such implementations, the pilot hole can be drilled using force and/or torque sensing as described herein.

In an operational block 130, the method 100 further comprises monitoring at least one signal indicative of insertion of the screw portion 212 into the tissue during said driving. In an operational block 140, the method further comprises stopping said driving in response to the at least one signal being indicative of a predetermined insertion of the screw portion 212. In certain implementations, the at least one signal is indicative of an insertion depth of the screw portion 212 into the tissue, while in certain other implementations, the at least one signal is indicative of a proximity of the screw portion 212 to the cavity (e.g., a distance between the screw portion 212 and an inner wall of the vestibular cavity). By monitoring the at least one signal during the driving of the screw portion 212 and stopping the driving in response to the at least one signal being indicative of a predetermined insertion of the screw portion 212, certain implementations described herein allow the insertion to be stopped before the electrode 210 breaches the vestibule (see, e.g., FIG. 1A which includes a white shape indicative of an access path that can be drilled near to the vestibule between critical structures and stopping before breaching into the vestibular cavity).

FIGS. 3A-3C schematically illustrate axial diagrams of the cochleovestibular system and an apparatus 400 comprising an electrode 210 with various example driving systems 300 in accordance with certain implementations described herein. In each of FIGS. 3A-3C, the electrode 210 (e.g., bone screw) is implanted with the screw portion 212 inserted within bony tissue in proximity to the oval window but not breaching the inner surface of the vestibule.

Each example driving system 300 of FIGS. 3A-3C comprises a motor 310 and a portion 320 configured to be in mechanical communication with the motor 310 and in mechanical communication with (e.g., to mate with) at least a portion of the electrode 210 (e.g., the connection portion 214). For example, the electrode 210 can comprise a recess (e.g., slot; cylindrical hole having a polygonal cross-section in a plane perpendicular to an axial direction of the hole) and the portion 320 can comprise an extension (e.g., blade; cylindrical protrusion having a polygonal cross-section in a plane perpendicular to an axial direction of the protrusion) configured to fit within the recess. For another example, the portion 320 can comprise a recess (e.g., hole) and the electrode 210 can comprise an extension (e.g., protrusion) configured to fit within (e.g., mate with) the recess. The motor 310 of the example driving system 300 is configured to generate a force and/or torque and the portion 320 is configured to impart the force and/or torque to the screw portion 212 of the electrode 210 so as to drive the screw portion 212 into the tissue.

FIG. 3A schematically illustrates an example driving system 300 in which the at least one signal comprises at least one feedback signal indicative of the force and/or torque being applied to the screw portion 212 during said driving. In certain such implementations, the amount of force and/or torque being applied to the screw portion 212 is dependent upon the level of insertion of the screw portion 212. For example, smaller amounts of force and/or torque can correspond to smaller levels of insertion (e.g., due to relatively small amounts of resistance and/or friction created by rotating the screw portion 210 within the tissue when the screw portion 212 has a smaller insertion depth) while larger amounts of force and/or torque can correspond to higher levels of insertion (e.g., due to relatively larger amounts of resistance and/or friction created by rotating the screw portion 210 within the tissue when the screw portion 212 has a larger insertion depth). The force and/or torque sensing of certain example driving systems 300 compatible with certain such implementations described herein can be similar to that of force and/or torque sensing drilling systems designed to drill a cochleostomy for insertion of a cochlear electrode (see, e.g., X. Du et al., “Robustness analysis of a smart surgical drill for cochleostomy,” Int. J. Med. Robot., Vol. 9 (1) pp. 119-126.21 (2013); P. Brett et al., “Feasibility Study of a Hand Guided Robotic Drill for Cochleostomy,” Biomed. Res. Int'l, Vol. 2014, Article ID 656325, 7 pages, https://dx.doi.org/10.1155/2014/656325).

As schematically illustrated by FIG. 3A, the example driving system 300 can further comprise a monitoring module 330 configured to monitor the force and/or torque applied by the driving system 300 to the screw portion 212 and to generate the at least one signal indicative of the applied force and/or torque. For example, the monitoring module 330 can be operatively coupled to the motor 310 and can be configured to detect the force and/or torque outputted by the motor 310. The example driving system 300 of FIG. 3A can further comprise a controller 340 configured to receive the at least one signal from the monitoring module 330 and to generate control signals configured to control the motor 310 (e.g., turn the motor 310 on and/or off). The controller 340 can be configured to compare the applied force and/or torque indicated by the at least one signal to a predetermined maximum force and/or torque value indicative of a predetermined maximum insertion depth of the screw portion 212 (e.g., a predetermined minimum distance between the screw portion 212 and the inner wall of the vestibular cavity). For example, for a vestibular electrode 210 being implanted with the screw portion 212 within the bony tissue in proximity to the oval window, the maximum insertion depth can correspond to the screw portion 212 not breaching the inner surface of the vestibular cavity, and the control signals can turn off the motor 310 to stop the insertion from proceeding any further. In this way, the at least one signal indicative of the applied force and/or torque is used as real-time feedback signal to ensure that breach of the vestibular cavity does not occur.

FIG. 3B schematically illustrates an example driving system 300 in which the at least one signal comprises at least one feedback signal indicative of an electrical impedance between the electrode 210 and a predetermined portion of the recipient's body (e.g., tissue; bodily fluids; the perilymph) during said driving. In certain such implementations, the amount of electrical impedance between the electrode 210 and the body is dependent upon the level of insertion of the screw portion 212. For example, larger amounts of electrical impedance can correspond to smaller levels of insertion (e.g., due to relatively smaller size of the electrical pathway between the electrode 210 and the body when the screw portion 212 has a smaller insertion depth or is farther away from the perilymph) while smaller amounts of electrical impedance can correspond to higher levels of insertion (e.g., due to relatively larger size of the electrical pathway between the electrode 210 and the body when the screw portion 212 has a larger insertion depth or is closer to the perilymph). For example, the reduction of the electrical impedance as the screw portion 212 nears the vestibule can be a sensitive measurement of the proximity of the screw portion 212 to the inner wall of the vestibule. In certain implementations, the at least one signal can comprise a complex electrical impedance signal and/or an electrochemical impedance spectroscopic signal.

As schematically illustrated by FIG. 3B, the example driving system 300 can further comprise electrical conduits 350a,b (e.g., wires) in electrical communication with the electrode 210 and the body, respectively. The example driving system 300 can further comprise a monitoring module 360 in electrical communication with the electrical conduits 350a,b and configured to monitor the electrical impedance between the electrode 210 and the body and to generate the at least one signal indicative of the detected electrical impedance. The example driving system 300 of FIG. 3B can further comprise a controller 340 configured to receive the at least one signal from the monitoring module 360 and to generate control signals configured to control the motor 310 (e.g., turn the motor 310 on and/or off). The controller 340 can be configured to compare the detected electrical impedance indicated by the at least one signal to a predetermined minimum electrical impedance value (e.g., threshold) or gradient indicative of a predetermined maximum insertion depth of the screw portion 212 (e.g., a predetermined minimum distance between the screw portion 212 and the inner wall of the vestibular cavity). For example, for a vestibular electrode 210 being implanted with the screw portion 212 within the bony tissue in proximity to the oval window, the maximum insertion depth can correspond to the screw portion 212 not breaching the inner surface of the vestibular cavity, and the control signals can turn off the motor 310 to stop the insertion from proceeding any further. In this way, the at least one signal indicative of the detected electrical impedance is used as real-time feedback signal to ensure that breach of the vestibular cavity does not occur.

FIG. 3C schematically illustrates an example driving system 300 in which the at least one signal comprises at least one feedback signal indicative of the force and/or torque being applied to the screw portion 212 during said driving and at least one feedback signal indicative of an electrical impedance between the electrode 210 and a predetermined portion of the recipient's body (e.g., tissue; bodily fluids; the perilymph) during said driving. As schematically illustrated by FIG. 3C, the example driving system 300 can further comprise the monitoring module 330 of FIG. 3A and the electrical conduits 350a,b (e.g., wires) and monitoring module 360 of FIG. 3B. The controller 340 of FIG. 3B can be configured to receive both sets of feedback signals from the monitoring modules 330, 360 and to generate control signals configured to control the motor 310 (e.g., turn the motor 310 on and/or off). By using both sets of feedback signals, certain such implementations can better ensure that breach of the vestibular cavity does not occur.

In certain implementations, the example driving system 300 is further configured to receive at least one signal comprising at least one physiological and/or electrophysiological signal indicative of a response by the recipient to electrical stimulation applied to the electrode 210 (e.g., in addition to the signals indicative of the applied force and/or torque and/or the signals indicative of the electrical impedance). For example, the at least one signal can be indicative of local neural response or response from higher in the recipient's neural pathway. By indicating physiological and/or electrophysiological responses to the electrical stimulation applied to the electrode 210, certain implementations can provide the practitioner indications that the electrode 210 has been inserted to a sufficient depth and does not have to be inserted further.

FIGS. 4A-4B, 5A-5D, and 6A-6D schematically illustrate example apparatus 400 (e.g., electrode 210) compatible with certain implementations described herein. The apparatus 400 comprises a bone screw portion 410 (e.g., self-drilling and/or self-tapping screw portion 212) configured to be rotated about an axial direction 420 to drill and/or tap into bone tissue (e.g., outside and adjacent to a vestibular cavity of a recipient). The apparatus 400 further comprises a head portion 430 configured to be mechanically engaged (e.g., by a driving system 300) and rotated about the axial direction 420 to drill and/or tap the bone screw portion 410 into the bone tissue. The combination of the bone screw portion 410 and the head portion 430 is referred to herein as the fixture 435.

The apparatus 400 of certain implementations further comprises an electrically conductive connector portion 440 (e.g., an electrically conductive wire having an electrical insulating sheath) configured to be in electrical communication with an electrically conductive conduit 450 while the bone screw portion 410 and the head portion 430 (e.g., the fixture 435) are rotated about the axial direction 420. In certain implementations, at least one of the bone screw portion 410, the head portion 430, and the connector portion 440 comprises an electrically conductive and biocompatible material (e.g., titanium; titanium alloy).

As schematically illustrated by FIGS. 4A-4B, the head portion 430 comprises a recess (e.g., slot or hole having a polygonal cross-section in a plane perpendicular to the axial direction 420) configured to be mechanically engaged and rotated by a portion of the driving system 300 (e.g., a blade or protrusion having a polygonal cross-section in a plane perpendicular to the axial direction 420) such that the bone screw portion 410 and the head portion 430 (e.g., the fixture 435) are rotated about the axial direction 420. In certain other implementations, the head portion 430 comprises an extension (e.g., protrusion) configured to fit (e.g., mate) with a corresponding recess (e.g., hole) of the driving system 300.

In certain implementations, the head portion 430 has a width in a plane substantially perpendicular to the axial direction 420 that is larger than a width of the bone screw portion 410 in a plane substantially perpendicular to the axial direction 420. In this way, the head portion 430 can be configured to provide a physical limit (e.g., a maximum insertion depth; equal to a length of the bone screw portion 410) for implantation of the apparatus 400 into the tissue. In certain implementations, the physical limit is less than a thickness of the bone tissue at the implantation site (e.g., measured from pre-operative imaging). For example, the head portion 430 can comprise a stopper nut or a set of removable spacers that leave only the desired length of the bone screw portion 410 protruding, thereby preventing the bone screw portion 410 from penetrating into a nearby cavity (e.g., vestibule).

In certain implementations, as schematically illustrated by the right side of FIG. 4A, the connector portion 440 is between the bone screw portion 410 and the head portion 430. The connector portion 440 is configured to be in rotatable communication with the conduit 450 such that electrical connectivity between the connector portion 440 and the conduit 450 is maintained while the apparatus 400 is rotated about the axial direction 420. For example, as schematically illustrated by the left side of FIG. 4A, the conduit 450 can comprise a substantially circular clip portion 452 comprising an electrically conductive material (e.g., titanium; titanium alloy) configured to fit (e.g., snap) onto a substantially circular perimeter of the connector portion 440 (e.g., a waist portion of the electrode 210) schematically illustrated in the right panel of FIG. 4A. The conduit 450 of FIG. 4A further comprises at least one electrically conductive protrusion 454 extending inwardly from the clip portion 452 and an electrically conductive wire 456 (e.g., flexible wire; wire 350a) in electrical communication with the at least one protrusion 454 (e.g., via an electrically conductive and substantially circular clip portion 452). The at least one protrusion 454 can be spring-loaded such that electrical communication is maintained (e.g., at multiple points) between the conduit 450 and the connector portion 440 while the apparatus 400 rotated (e.g., the conduit 450 is configured to slip around the connector portion 440 during rotation of the electrode 210). In certain implementations, the conduit 450 is configured to be in electrical communication with the driving system 300 to provide electrical impedance measurements for controlling the driving system 300. In certain such implementations, the conduit 450 is also in electrical communication with an electrical stimulation system configured to provide stimulation signals to the apparatus 400 (e.g., electrode 210). To insulate the head portion 430 of the apparatus 400 from the surrounding tissue, an electrically insulating (e.g., silicone) cap can be placed over (e.g., pressed onto) the head portion 430 and the connection portion 440.

In certain implementations, as schematically illustrated by FIG. 4B, the connector portion 440 comprises an outer perimeter of the head portion 430 and the electrically conductive conduit 450 comprises at least one clip 370 at a distal end of the portion 320 of the driving system 300. The at least one clip 370 can comprise a plurality (e.g., three or more) of tines (e.g., leaf springs) configured to hold the head portion 430 with a spring-loaded force with the connector portion 440 while maintaining electrical connectivity between the connector portion 440 and the conduit 450 during rotation of the apparatus 400 about the axial direction 420. For example, the at least one leaf spring 370 can comprise an electrically conductive material (e.g., titanium; titanium alloy) configured to fit (e.g., snap; clasp) onto the outer perimeter of the head portion 430. In certain implementations, the conduit 450 is configured to be in electrical communication with the driving system 300 to provide electrical impedance measurements for controlling the driving system 300. In certain such implementations, the conduit 450 is configured to be removed from the connector portion 440 with the driving system 300 once the apparatus 400 is implanted.

In certain implementations, the apparatus 400 can further comprise a fluid conduit extending from a proximal end of the head portion 430 to a distal end of the bone screw portion 410. For example, the fluid conduit can be configured to deliver a drug through the apparatus 400 (e.g., delivered via a needle introduced into an input port of the fluid conduit) to a target region at or near an output port of the fluid conduit (e.g., the endosteum). For another example, the apparatus 400 can comprise a cavity and/or sponge in fluid communication with the fluid conduit and comprising the drug to be delivered through an output port of the fluid conduit.

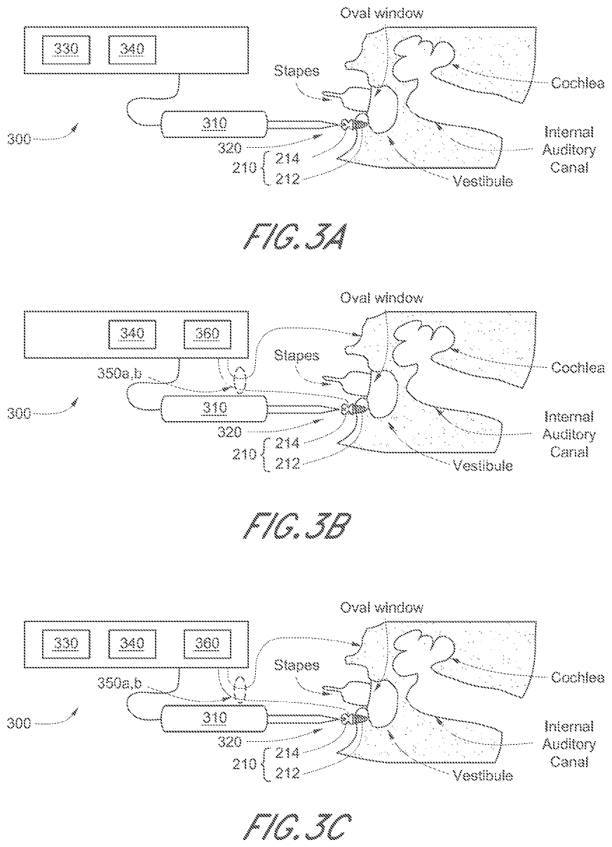

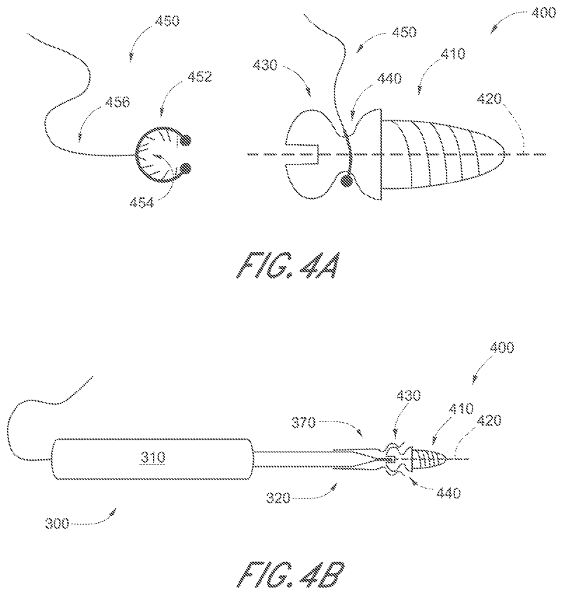

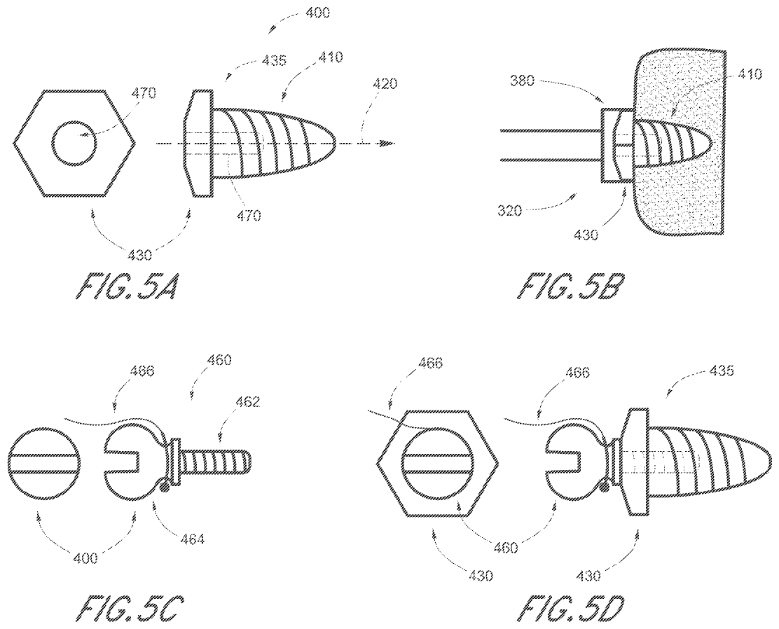

FIGS. 5A-5D schematically illustrate an example apparatus 400 comprising multiple components that are mechanically coupled to one another during the implantation process in accordance with certain implementations described herein. FIG. 5A schematically illustrates two views of an example apparatus 400 comprising a fixture 435 (e.g., the bone screw portion 410 and head portion 430) in accordance with certain implementations described herein. The head portion 430 has an outer perimeter configured to be mechanically engaged and rotated by a portion of the driving system 300 such that the fixture 435 (e.g., the bone screw portion 410 and the head portion 430) is rotated about the axial direction 420. For example, as schematically illustrated by FIGS. 5A and 5B, the head portion 430 can have a polygonal perimeter (e.g., hexagonal) configured to be mechanically engaged (e.g., held) and rotated by a socket 380 at a distal end of the portion 320 of the driving system 300. In certain implementations, the outer perimeter (e.g., connector portion 440) is further configured to be in electrical communication with the portion of the driving system 300 to serve as a pathway for electrical impedance measurements from the apparatus 400 while the apparatus 400 is being implanted. For example, the socket 380 can be configured to hold the head portion 430 and to provide electrical connectivity between the driving system 300 and the apparatus 400 such that the driving system 300 can make electrical impedance measurements while driving the apparatus 400 into the tissue. In certain implementations, the outer perimeter of the head portion 430 is configured to be disengaged from the portion of the driving system 300 (e.g., socket 380) once the apparatus 400 has been implanted.

FIG. 5C schematically illustrates two views of an example electrically conductive connector 460 of the apparatus 400 and configured to provide electrical conductivity to the apparatus 400 once the apparatus 400 is implanted in accordance with certain implementations described herein. For example, as schematically illustrated by FIG. 5C, the connector 460 comprises a connector mating portion 462 (e.g., a machined threaded screw; a spring-loaded fitting) and an electrically conductive second head portion 464 configured to be in electrical communication with an electrically conductive conduit 466. The fixture 435 (e.g., the head portion 430 and/or the bone screw portion 410) can comprise a corresponding fixture mating portion 470 configured to mate with the connector mating portion 462. For example, as schematically illustrated by FIGS. 5A and 5B, the fixture mating portion 470 can comprise a recess (e.g., a threaded hole; a machined tapped hole; an unthreaded or untapped hole) extending along the axial direction 420 through the head portion 430 and at least partially through the bone screw portion 410, and the fixture mating portion 470 can be configured to mate with (e.g., be screwed into; press-fit into; snapped into) the connector mating portion 462 such that the connector 460 is in electrical communication with the fixture 435. In certain other implementations, the connector mating portion 462 and the corresponding fixture mating portion 470 can comprise corresponding recesses, protrusions, leaf springs, or other complementary structures configured to mechanically engage (e.g., screw; fit; snap) together to place the connector 460 in electrical communication with the fixture 435. In certain implementations, the fixture 435 and the connector 460 are configured to be positioned and implanted by a single driving system 300 (e.g., with different structures on a distal end of the portion 320), while in certain other implementations, these components are configured to be positioned and implanted by different driving systems 300 tailored for implantation of the corresponding component of the apparatus 400.

FIG. 5D schematically illustrates two views of the connector 460 in mechanical and electrical communication with the fixture 435. In certain implementations, the second head portion 464 comprises at least one recess and/or protrusion configured to mechanically engage a corresponding at least one protrusion and/or recess of the driving system 300 such that the driving system 300 can hold and rotate the connector 460 to screw the connector mating portion 462 into the hole of the fixture mating portion 470. In this way, the connector 460 can be mechanically coupled to the fixture 435, and the driving system 300 can be withdrawn. Once the connector 460 is in mechanical and electrical communication with the fixture 435, the stimulation system can provide electrical stimulation signals to the apparatus 400 via the conduit 466.

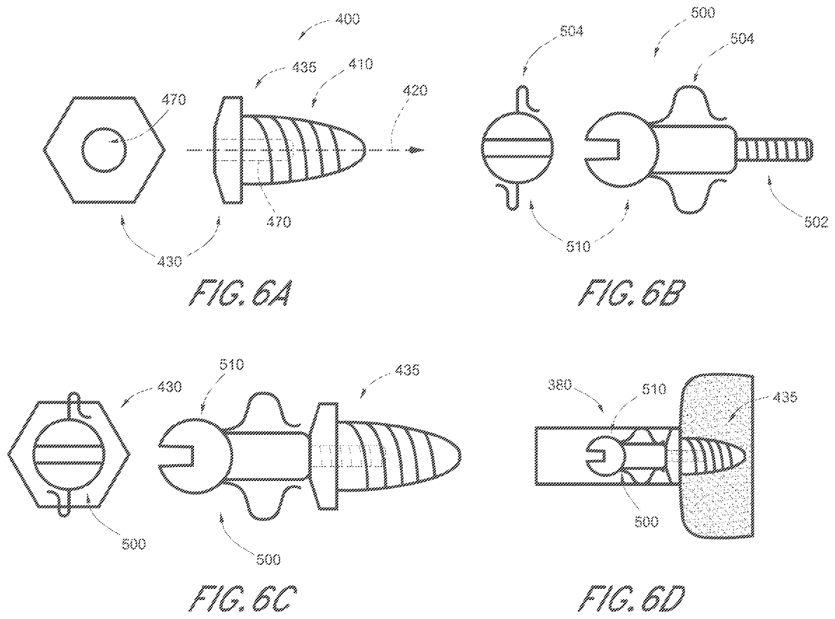

FIGS. 6A-6D schematically illustrate another example apparatus 400 configured to provide additional electrical conductivity between the driving system 300 and the apparatus 400 in accordance with certain implementations described herein. FIG. 6A schematically illustrates two views of the apparatus 400 comprising a bone screw portion 410, a head portion 430, and a fixture mating portion 470 (e.g., as described herein with regard to FIG. 5A) in accordance with certain implementations described herein.

FIG. 6B schematically illustrates two views of an example electrically conductive first component 500 of the apparatus 400 configured to provide additional electrical conductivity to the apparatus 400 while the apparatus 400 is being implanted in accordance with certain implementations described herein. FIG. 6C schematically illustrates two views of the first component 500 mated with the fixture 435 in accordance with certain implementations described herein. For example, the first component 500 can comprise a first mating portion 502 (e.g., a machined threaded screw; a spring-loaded fitting) configured to mechanically engage (e.g., mate with; screw into; press-fit or snap within) a corresponding mating portion (e.g., recess; threaded hole; unthreaded hole; the fixture mating portion 470) of the fixture 435 and configured to be disengaged (e.g., removed) from the corresponding mating portion after the apparatus 400 is implanted. The first component 500 can further comprise at least one electrically conductive leaf spring 504 configured to be in electrical communication with a portion of the driving system 300 while the apparatus 400 is being implanted. For example, as schematically illustrated by FIG. 6D, a socket 380 of the driving system 300 can be configured to mechanically engage both the perimeter of the head portion 430 and the at least one leaf spring 504 and to be in electrical communication with the at least one leaf spring 504. In this way, the first component 500 can provide electrical communication between the driving system 300 and the apparatus 400 via the at least one leaf spring 504 (e.g., in addition to electrical communication via the perimeter of the head portion 430).

In certain implementations, the first component 500 further comprises a third head portion 510 configured to be mechanically engaged (e.g., by the driving system 300) and removed along with the rest of the first component 500 from the implanted fixture 435. For example, as schematically illustrated by FIGS. 6B-6D, the third head portion 510 can comprise at least one recess and/or protrusion configured to mechanically engage a corresponding at least one protrusion and/or recess of the driving system 300 such that the driving system 300 can hold and remove the first component 500 from the fixture mating portion 470 (e.g., rotate the first component 500 to unscrew the threaded screw of the first mating portion 502 from the threaded hole of the fixture mating portion 470). In this way, the first component 500 can be decoupled from the fixture 435, and the first component 500 can be withdrawn from the fixture 435. In certain implementations, the first connector 500 is configured to be removed from the fixture 435 by the same driving system 300 that positions and implants the first connector 500 and fixture 435 (e.g., using different structures on a distal end of the portion 320), while in certain other implementations, the first connector 500 is configured to be removed by a different driving system 300 tailored for such removal.

In certain implementations, the apparatus 400 further comprises a second component 600 comprising a second mating portion 602 (e.g., a machined threaded screw; a spring-loaded fitting; at least one protrusion and/or recess) configured to mechanically engage (e.g., mate with; screw into; press-fit or snap within) the corresponding mating portion (e.g., threaded hole; unthreaded hole; fixture mating portion 470; at least one recess and/or protrusion) after the fixture 435 has been implanted. For example, the second component 600 can comprise the electrically conductive connector 460 (e.g., as described herein with regard to FIGS. 5C and 5D), and the second mating portion 602 can comprise the connector mating portion 462 (e.g., a second threaded screw) configured to mate with the fixture mating portion 470 (e.g., screwed into the threaded hole) after the first mating portion 502 (e.g., a machined threaded screw) is removed from the fixture mating portion 470 (e.g., unscrewed from the threaded hole).

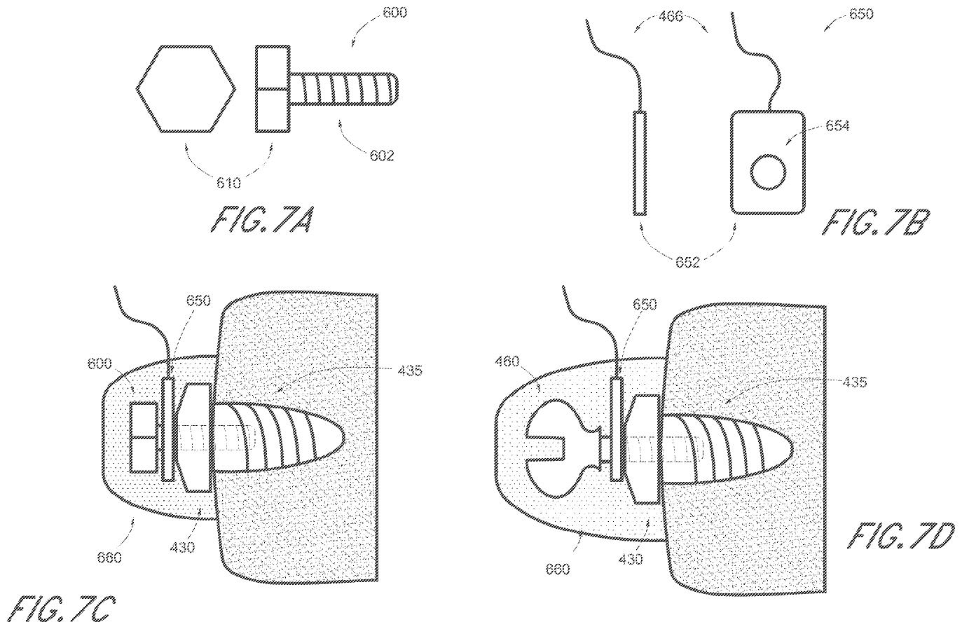

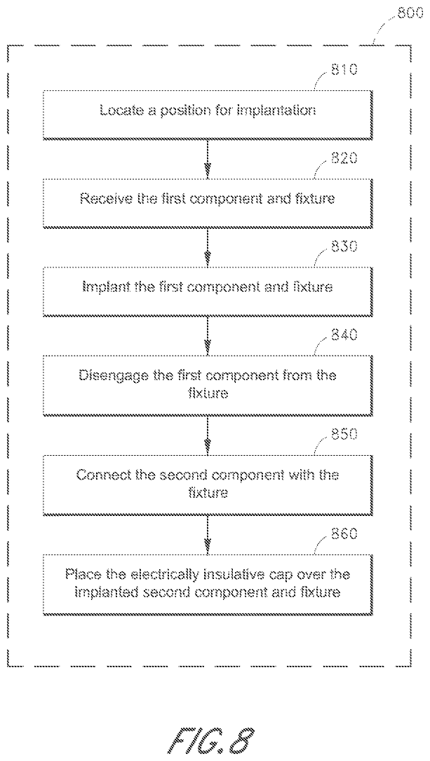

FIGS. 7A-7D schematically illustrate another example apparatus 400 comprising a second component 600 in accordance with certain implementations described herein. As shown in the two views of FIG. 7A, the second component 600 can comprise a second head portion 610 configured to be mechanically engaged and rotated by the portion 320 of the driving system 300. For example, the second head portion 610 can have a hexagonal or other polygonal perimeter configured to be mechanically engaged (e.g., held) and rotated by a socket 380 at a distal end of the portion 320 of the driving system 300. The second head portion 610 further can comprise the second mating portion 602 (e.g., a machined threaded screw) configured to mechanically engage (e.g., mate with; fit or snap within) the corresponding mating portion (e.g., threaded hole; the fixture mating portion 470) of the implanted fixture 435 after the first component 500 is removed from the corresponding mating portion (e.g., unscrewed from the threaded hole).

In certain implementations, the apparatus 400 further comprises a third component 650 configured to be sandwiched between the head portion 430 and the second head portion 610 and at least partially encircling the second mating portion 602 mechanically engaged with the corresponding mating portion of the implanted fixture 435. For example, as shown in the two views of FIG. 7B, the third component 650 can comprise an electrically conductive portion 652 (e.g., planar portion) in electrical communication with an electrically conductive conduit 466 in electrical communication with a stimulation system. The third component 650 can further comprise a through-hole 654 configured to have the second mating portion 602 (e.g., threaded screw) extending therethrough. In certain implementations, the second component 600 and the third component 650 are configured to be positioned and implanted by a single driving system 300 (e.g., the same driving system 300 that positions and implants the fixture 435 (e.g., with different structures on a distal end of the portion 320), while in certain other implementations, the second component 600 and the third component are configured to be positioned and implanted by the same driving system 300 but that is different from the driving system 300 that positions and implants the fixture 435.

FIG. 7C schematically illustrates the second component 600 of FIG. 7A and the third component 650 of FIG. 7B in mechanical and electrical communication with the fixture 435 of FIGS. 5A and 6A implanted to bone tissue. FIG. 7D schematically illustrates the second component 600 comprising the connector 460 of FIG. 5C and the third component 650 of FIG. 7B in mechanical and electrical communication with the fixture 435 of FIGS. 5A and 6A implanted to bone tissue. As schematically illustrated by FIGS. 7C and 7D, in certain implementations, the apparatus further comprises and electrically insulating cap 660 (e.g., silicone) configured to electrically insulate at least the head portion 430 from surrounding biological materials (e.g., in the middle ear region).

FIG. 8 is a flow diagram of an example method 800 for implanting the apparatus 400 in accordance with certain implementations described herein. In an operational block 810, the method 800 comprises locating a position for implantation of the apparatus 400. For example, for implanting a vestibular or cochlear electrode, the position can be on an outer surface of the cochlear bone.

In an operational block 820, the method 800 further comprises receiving the apparatus 400 comprising the first component 500 in mechanical and electrical communication with the fixture 435. For example, said receiving can comprise receiving the first component 500 separately from receiving the fixture 435 and assembling the first component 500 to the fixture 435. In certain implementations, the mechanical coupling between the first component 500 and the fixture 435 is sufficiently strong to keep the first component 500 and the fixture 435 in mechanical and electrical communication with one another during implantation of the fixture 435 but is sufficiently weak that the first component 500 can be removed from the fixture 435 after implantation of the fixture 435.

In an operational block 830, the method 800 further comprises implanting the combination of the first component 500 and the fixture 435 while monitoring the at least one signal indicative of the force and/or torque applied by the driving system and/or indicative of the electrical impedance between the fixture 435 and the body (e.g., using a driving system 300 as described herein). In certain implementations, the implantation is completed once the at least one signal indicates that the predetermined insertion has been achieved.

In an operational block 840, the method 800 further comprises disengaging the first component 500 from the fixture 435 while the fixture 435 remains implanted. For example, the fixture 435 can be held in place by a socket while the first component 500 is unscrewed from the fixture mating portion 470.

In an operational block 850, the method 800 further comprises connecting the second component 600 and/or third component 650 with the fixture 435 such that the second component 600 and/or third component 650 is in mechanical and electrical communication with the fixture 435. For example, the second component 600 (e.g., connector 460) can be inserted (e.g., screwed) into the fixture coupling portion 470 with the third component 650 sandwiched between the second head portion 610 (e.g., second head portion 464) and the head portion 430. In an operational block 860, the method 800 can further comprise placing the electrically insulative cap 660 over the second component 600 and the fixture 435.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having at least a portion of the received power available for use by the implanted device during time periods in which the at least one power storage device of the implanted device unable to provide electrical power for operation of the implantable medical device.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. A method comprising:
providing an electrode configured to be implanted within a body portion of a recipient in tissue at a target implantation location, the electrode comprising a screw portion;
driving the screw portion into the tissue at the target implantation location;
monitoring at least one signal indicative of insertion of the screw portion into the tissue during said driving; and
stopping said driving in response to the at least one signal being indicative of a predetermined insertion of the screw portion.

2. The method of claim 1, wherein the body portion comprises a middle ear region and/or an inner ear region of the recipient.

3. The method of claim 2, wherein the tissue at the target implantation location is bone tissue outside and adjacent to a vestibular cavity, and the target implantation location is sufficiently close to the otoliths and/or the vestibular nerve such that the otoliths and/or vestibular nerve are stimulated by a predetermined voltage and/or current applied to the electrode.

4. The method of claim 3, wherein the predetermined insertion corresponds to a predetermined distance between the screw portion and an inner wall of the vestibular cavity.

5. The method of claim 2, wherein the tissue at the target implantation location is on a surface portion of a cochlea within the middle ear region, such that nerves within the cochlear are stimulated by a predetermined electrical current and/or voltage applied to the electrode.

6. The method of claim 1, further comprising drilling a channel towards the body portion along a pre-operatively determined path configured to avoid damaging predetermined tissue portions and said providing the electrode comprises moving the electrode through the channel and positioning the electrode adjacent to the tissue at the target implantation location.

7. The method of claim 6, wherein the body portion comprises a middle ear region and/or an inner ear region of the recipient and said drilling does not damage any semicircular canal, facial nerve, and/or cochleovestibular nerve of the recipient.

8. The method of claim 1, wherein the at least one signal comprises at least one feedback signal indicative of a force and/or torque being applied to the screw portion during said driving.

9. The method of claim 1, wherein the at least one signal comprises at least one feedback signal indicative of an electrical impedance between the electrode and a predetermined tissue portion of the recipient.

10. The method of claim 1, wherein said tissue at the target implantation location comprises a previously-drilled pilot hole configured to receive the screw portion and said driving comprises inserting the screw portion into the pilot hole.

11. The method of claim 1, wherein the at least one signal comprises at least one physiological and/or electrophysiological signal indicative of a response by the recipient to electrical stimulation applied to the electrode.

12. An apparatus comprising:
a bone screw portion configured to be rotated about an axial direction to drill and/or tap into bone tissue outside and adjacent to a vestibular cavity of a recipient;
a head portion configured to be mechanically engaged and rotated about the axial direction to drill and/or tap the bone screw portion into the bone tissue; and
an electrically conductive connector portion configured to be in electrical communication with an electrically conductive conduit while the bone screw portion and the head portion are rotated about the axial direction.

13. The apparatus of claim 12, wherein the connector portion is between the bone screw portion and the head portion, the connector portion configured to be in rotatable communication with a clip portion of the conduit.

14. The apparatus of claim 12, wherein the head portion comprises a recess configured to be mechanically engaged and rotated by a portion of a driving system such that the bone screw portion and the head portion are rotated about the axial direction.

15. The apparatus of claim 12, wherein a perimeter of the head portion is configured to be mechanically engaged and rotated by a portion of a driving system such that the bone screw portion and the head portion are rotated about the axial direction.

16. The apparatus of claim 15, wherein the apparatus further comprises a threaded hole extending along the axial direction through the head portion and at least partially through the bone screw portion, the apparatus further comprises:
a first component comprising:
a first threaded screw within the threaded hole and configured to be removed from the threaded hole; and
at least one electrically conductive leaf spring configured to be in electrical communication with a portion of the driving system when the perimeter is mechanically engaged by the portion of the driving system; and
a second component comprising:
a second head portion; and
a second threaded screw configured to be screwed into the threaded hole after the first threaded screw is removed from the threaded hole.

17. The apparatus of claim 16, further comprising a third component configured to be sandwiched between the head portion and the second head portion and at least partially encircling the second threaded screw in the threaded hole.

18. The apparatus of claim 15, wherein the apparatus further comprises:
a recess extending along the axial direction through the head portion and at least partially through the bone screw portion;
a first component comprising:
a first spring-loaded fitting that is press-fit within the recess and configured to be removed from the recess; and
at least one electrically conductive leaf spring configured to be in electrical communication with a portion of the driving system when the perimeter is mechanically engaged by the portion of the driving system; and
a second component comprising:
a second head portion; and
a second spring-loaded fitting configured to be press-fit into the recess after the first spring-loaded fitting is removed from the recess.

19. The apparatus of claim 12, further comprising an electrically insulating cap configured to electrically insulate at least the head portion from surrounding biological materials.

20. A system comprising:
an electrode comprising a screw portion configured to be inserted into bone tissue of a recipient; and
a controller in electrical communication with the electrode, the controller configured to monitor a status of the screw portion during insertion of the screw portion into the bone tissue and to transmit electrical stimulation signals to the electrode after the screw portion is inserted into the bone tissue.

21. The system of claim 20, wherein the bone tissue is outside and adjacent to a vestibular cavity of the recipient, the screw portion configured to not extend into the vestibular cavity when implanted.

22. The system of claim 20, wherein the screw portion is configured to self-drill and/or self-tap into the bone tissue.

23. The system of claim 20, further comprising a flexible electrical conduit in electrical communication with the electrode and the controller during said insertion of the screw portion and after the screw portion is inserted.
